(19) United States
(12) Patent Application Publication
(10) Pub. No.: US 2003/0032675 A1
(43) Pub. Date: Feb. 13, 2003

(54) Manufacture of thyroid hormone tablets having consistent active moiety amounts

(76) Inventors: G. Andrew Franz, St. Louis, MO (US); Elaine A. Strauss, Seminole, FL (US); Philip A. DiMenna, St. Petersburg, FL (US); Rocco L. Gemma, Dover, OH (US); Matthew Smith, West Chester, OH (US); Scott C. Vaaler, Cincinnati, OH (US); Floyd Meredith, Cincinnati, OH (US)

Correspondence Address: Edwards & Angell, LLP, Suite 514, 600 Corporate Drive, Ft. Lauderdale, FL, 33334, US

(21) Appl. No.: 10/076,731

(22) Filed: Feb. 15, 2002

Related U.S. Application Data
(60) Provisional application No. 60/269,009, filed on Feb. 15, 2001.
(60) Provisional application No. 60/268,998, filed on Feb. 15, 2001.

(51) Int. Cl. D04H 1/00; B27N 3/00; B01F 15/02; A61K 31/198

(52) U.S. Cl. 514/567; 264/109; 366/131

(57) ABSTRACT

An apparatus for the transport of thyroid hormone drug formulations from a blender to a tablet press using mass flow of the formulation in order to maintain consistent tablet compositions during manufacture of the tablets.

### BACKGROUND OF INVENTION

This invention relates to an improved method of manufacturing thyroid hormone preparations of levothyroxine sodium, liothyronine sodium and similar products in tablet form. Such tablets are pharmaceutical preparations useful to the treatment of hypothyroidism and thyroid hormone replacement therapy in mammals, for example, humans and dogs.

Thyroid hormone drugs are natural or synthetic preparations containing tetraiodothyronine (T₄, levothyroxine) sodium or triiodothyronine (T₃, liothyronine) sodium or both. T₄ and T₃ are produced in the human thyroid gland by the iodination and coupling of the amino acid tyrosine. T₄ contains four iodine atoms and is formed by the coupling of two molecules of diiodotyrosine (DIT). T₃ contains three atoms of iodine and is formed by the coupling of one molecule of DIT with one molecule of monoiodotyrosine (MIT). Both hormones are stored in the thyroid colloid as thyroglobulin. Thyroid hormone preparations belong to two categories: (1) natural hormonal preparations derived from animal thyroid, and (2) synthetic preparations. Natural preparations include desiccated thyroid and thyroglobulin.

Desiccated thyroid is derived from domesticated animals that are used for food by man (either beef or hog thyroid), and thyroglobulin is derived from thyroid glands of the hog. The United States Pharmacopoeia (USP) has standardized the total iodine content of natural preparations. Thyroid USP contains not less than (NLT) 0.17 percent and not more than (NMT) 0.23 percent iodine, and thyroglobulin contains not less than (NLT) 0.7 percent of organically bound iodine. Iodine content is only an indirect indicator of true hormonal biologic activity.

Synthetic forms for both T₄ and T₃ thyroid hormone are available from a number of producers. For example, liothyronine sodium (T₃) tablets are available from Jones Pharma, St Louis, Mo. under the trademark Cytomel (now King Pharmaceuticals, Inc.) Levothyroxine sodium (T₄) is available as the tradename Levoxyl from Jones Pharma (now King Pharmaceuticals, Inc.), as the tradename Synthroid from Knoll Pharmaceutical, Mt. Olive, N.J., and as the tradename Unithroid from Jerome Stevens Pharmaceuticals, Bohemia, N.Y. In addition a veterinarian preparation of levothyroxine sodium is available under the tradename Soloxine from Jones Pharma, St. Louis, Mo.

Hypothyroidism is a common condition. It has been reported in the United States Federal Register that Hypothyroidism has a prevalence of 0.5 percent to 1.3 percent in adults. In people over 60, the prevalence of primary hypothyroidism increases to 2.7 percent in men and 7.1 percent in women. Because congenital hypothyroidism may result in irreversible mental retardation, which can be avoided with early diagnosis and treatment, newborn screening for this disorder is mandatory in North America, Europe, and Japan.

Thyroid hormone replacement therapy can be a chronic, lifetime endeavor. The dosage is established for each patient individually. Generally, the initial dose is small. The amount is increased gradually until clinical evaluation and laboratory tests indicate that an optimal response has been achieved. The dose required to maintain this response is then continued. The age and general physical condition of the patient and the severity and duration of hypothyroid symptoms determine the initial dosage and the rate at which the dosage may be increased to the eventual maintenance level. It has been reported that the dosage increase should be very gradual in patients with myxedema or cardiovascular disease to prevent precipitation of angina, myocardial infarction, or stroke.

It is important that thyroid hormone treatment have the correct dosage. Both under treatment and over treatment can have deleterious health impacts. In the case of under treatment, a sub-optimal response and hypothyroidism could result. under treatment has also been reported to be a potential factor in decreased cardiac contractility and increased risk of coronary artery disease. Conversely, over treatment may result in toxic manifestations of hyperthyroidism such as cardiac pain, palpitations, or cardiac arrhythmia's. In patients with coronary heart disease, even a small increase in the dose of levothyroxine sodium may be hazardous in a particular

Hyperthyroidism is a known risk factor for osteoporosis. Several studies suggest that subclinical hyperthyroidism in premenopausal women receiving thyroid hormone drugs for replacement or suppressive therapy is associated with bone loss. To minimize the risk of osteoporosis, it is preferable that the dose be kept to the lowest effective dose.

Because of the risks associated with over treatment or under treatment with levothyroxine sodium, it is needed thyroid hormone products that are consistent in potency and bioavailability. Such consistency is best accomplished by manufacturing techniques that maintain consistent amounts of the active moiety during tablet manufacture.

### SUMMARY OF INVENTION

An improved apparatus for use in manufacturing of thyroid hormone tablets comprising a blender discharge section, a portable container section, a portable container discharge section, and a conical tableting machine inlet section wherein mass flow of the drug formulation is maintained in all sections of the apparatus. In one preferred embodiment, the portable container discharge section comprises a vent cone section. In another preferred embodiment the portable container discharge section comprises a vent cone section and a Y-branch section.

The improved apparatus is preferably used in manufacturing thyroid drug formulations of levothyroxine sodium or liothyronine sodium as the active moiety along with various excipients such as microcrystalline cellulose, calcium sulfate, gelatin, starch, stearic acid, lactose sucrose, talc. The improved apparatus is also preferably used in the manufacture of thyroid drug formulations comprising levothyroxine sodium and inert ingredients suitable for treating canine or feline hypothyroidism.

A process of manufacturing thyroid hormone preparations comprising blending of active moiety with excipients and inert ingredients to create a drug formulation, transporting the drug formulation from a blender to a tableting machine, preferably within a portable container and feeding the formulation to a tableting machine. Bulk mass flow is to be maintained during transfers from the interior of the blender, through intervening sections of the apparatus, to the tableting machine inlet.

### BRIEF DESCRIPTION OF DRAWINGS

FIG. 1a illustrates a blender discharge arrangement of one embodiment of the present invention;

FIG. 1b illustrates a blender discharge restricter of one embodiment of the present invention;

FIG. 1c illustrates an open dust collector vent for the blender discharge of one embodiment of the present invention;

FIG. 2a illustrates a portable container design of one embodiment of the present invention;

FIG. 2b illustrates a floating cylindrical connection of one embodiment of the present invention;

FIG. 3 illustrates a portable container discharge arrangement of one embodiment of the present invention;

FIG. 4 illustrates a vent cone and Y-branch design of one embodiment of the present invention;

FIG. 5 illustrates conical tablet press inlet hopper of one embodiment of the present invention;

FIG. 6 illustrates a blender filling arrangement of one embodiment of the present invention;

FIG. 7 illustrates a sifting segregation test;

FIG. 8 illustrates a fluidization segregation test.

FIG. 9defines storage container dimensions as used in Table 3 and Table 4.

### DETAILED DESCRIPTION

The present invention is directed to the manufacture of thyroid hormone drug tablets containing consistent amounts of the active moiety. As is more fully described below, the thrust of this invention is to ensure mass flow of the drug formulation from the blender to the tablet press or presses.

The various manufactures of thyroid hormone tablets formulate with varying amounts of excipients including tableting agents, binders, glidents, lubricants, disintegrants, colorants and flavorings. Such formulations can further be characterized as direct compression formulas, dry granulation formulas and wet granulation formulas. U.S. Pat. No. 5,955,105 to Mitra et al. describes parameters for making such formulations and is incorporated herein by reference in its entirety. Examples of direct compression formulas are illustrated as examples 1 to 16, 24 to 34 and 43 to 44 of '105 patent. After preparation of such formulas by blending, one batch at a time, the resultant compositions are directly fed to tableting machine for pressing into tablets.

Gravity feeding is typically used to transport the prepared direct compression formulation from the final blender to the tableting machine. For example, the contents of a formulation blender are transferred to a portable storage container. The portable storage container allows for accumulation of a batch when a tableting machine is unavailable or when sampling and assay of a batch is required before quality control release to tableting. The contents of such a portable storage container are, in turn, transported by gravity through chutes and vents into the tableting machine.

Design of the portable container, chutes and vents is critical to maintain consistency from one tablet to the next. The formulation batches are a blend of solid compositions of various shapes and sizes. Blending is used to achieve a measure of homogeneity. In particular the active thyroid moiety is desired to be evenly distributed throughout the batch. In a typical 400 kg batch, the amount of active moiety represents less than 1 kg of the total weight. For example, when producing 145 mg tablets with a 300 mcg dosage, approximately 0.8 kg of a 400 kg batch is the active moiety. In addition each tablet is to contain from 100% to 102.5% label claim potency (higher dosage levels may use a narrower 100% to 101% tolerance).

In order to achieve these high levels of consistency, particle segregation must be prevented and bulk solid mass flow must be maintained in material transfers between the blender and portable container and between the portable container and tableting machines. Segregation can either be sifting segregation or fluidization segregation.

Mass flow is defined as having all material flow simultaneously when material is removed from a blender or storage container, with uniform first-in-first-out flow. It contrast, funnel flow occurs when some material moves while other material remains stationary or stagnant. An example of funnel flow is ratholing that may occur with formulations and is often accompanied with bridging or arching.

Segregation testing can be performed for both sifting segregation and fluidization segregation. Sifting, which is a process by which smaller particles move through a matrix of larger ones, is the most common method of segregation. Four conditions must be present for sifting to occur:

1. A difference in particle size between the individual components. This ratio can be as low as 1.3 to 1. In general, the larger the ratio of particle sizes, the greater the tendency for particles to segregate by sifting.

2. A sufficiently large mean particle size. Sifting segregation can occur with a mean particle size in the 50 micron range and can become a dominant segregation mechanism if the mean particle size is above 100 microns.

3. Free flowing material. This allows the smaller particles to sift through the matrix of larger particles. With cohesive materials, the fine particles are bound to one another and do not enter the voids created by the coarse particles.

4. Interparticle motion. This can be caused during formation of a pile, by vibration, or by a velocity gradient across the flowing material.

All four of these conditions must be present for sifting segregation to occur. If any one of the four is absent, the mix will not segregate by this mechanism.

In materials having a range of particle sizes, the effect of sifting segregation may be significant in regard to product quality and handleability. As an illustration of this segregation mechanism. one might consider a pile formed by a falling stream or material. If sifting segregation takes place, the coarser particles will roll to the edges of this pile, whereas the finer particles will tend to sift through the larger particles and concentrate under the point of impact.

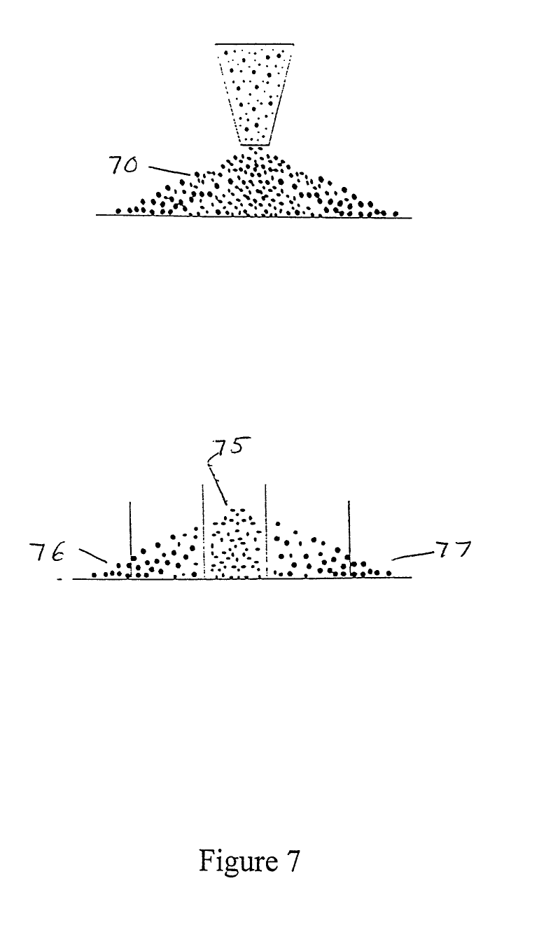

FIG. 7 illustrates the steps of testing for sifting segregation. In the first step, a conical pile 70 is carefully formed with the material being tested. The pile is then sectioned such that samples are taken from the center 75 and the periphery 76 and 77, as illustrated in the second step of FIG. 7.

In addition to sifting, fluidization can also cause segregation of blended particles. Fluidization can cause vertical segregation, i.e., horizontal layers of fines and coarse. Fine particles generally have a lower permeability than coarse particles and therefore tend to retain air longer. Thus on filling a hopper, the coarse particles are driven into the bed while the fine particles remain fluidized near the top surface. This can also occur after tumble blending if the material is fluidized during, blending. Air entrainment often develops in materials which contain a significant percentage of particles below 100 microns in size.

Fluidization segregation is likely to occur when fine materials are pneumatically conveyed, filled or discharged at high rates, or if gas counterflow is present.

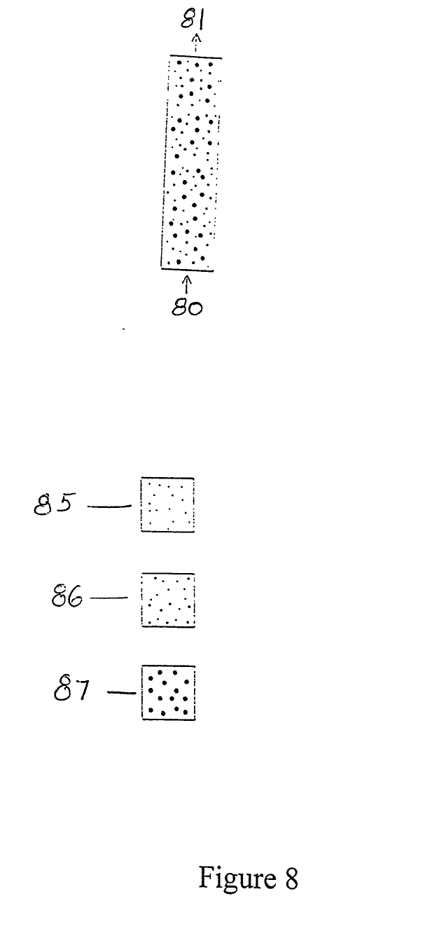

FIG. 8illustrates fluidization segregation testing. The fluidization segregation test is run by fluidizing a column of material by injecting air at the bottom 80, that in turn exits the fluidization column 81. After the column is thoroughly fluidized, the air is turned off and the material is allowed to deaerate. The column is then split into three equal sections: top 85, middle 86, and bottom 87.

### EXAMPLES

Segregation testing was performed on samples of the thyroid hormone drug formulation used in manufacturing Levoxyl brand levothyroxine sodium tablets. Tests were performed on direct compression formulations for dosage strengths of 25 mcg and 300 mcg. The results for sifting segregation testing are presented in Table 1 and for fluidization testing in Table 2:

TABLE 1
Sifting Segregation Test Results % Label Claim (sample 1,
sample 2)

| Sample | Center | Periphery |
|---|---|---|
| 25 mcg blend | 92.4, 91.2 | 88.8, 89.2 |
| 300 mcg blend | 95.2, 95.6 | 96.3, 95.9 |

TABLE 2
Fluidization Segregation Test Results % Label Claim (sample 1,
sample 2)

| Sample | Top | Middle | Bottom |
|---|---|---|---|
| 25 mcg blend | 93.6, 100.8 | 95.6, 95.6 | 95.2, 91.2 |
| 300 mcg blend | 95.7, 95.1 | 93.7, 93.0 | 93.1, 93.1 |

Based on these results. it is concluded that the potential for the materials to segregate, due to a sifting mechanism, is low and the potential for the materials to segregate, due to a fluidization mechanism, is moderate, and particularly high for the 25mcg dosage formulation.

Flow properties testing on these formulations indicate that they have moderate cohesive strength. One confinement yield strengths were 2.1 pounds per square foot (psf) and 6.2 psf for the 25 mcg and 300 mcg dosage formulations, respectively. The higher yield strength of the 300 mcg formulation indicates it has a tendency to form stable arches and ratholes if stored in a non-mass flow container. Hopper tests indicate that such a formulation requires a 3 to 5 inch diameter outlet to maintain reliable instantaneous flow and 6 inch diameter outlet after overnight storage at rest.

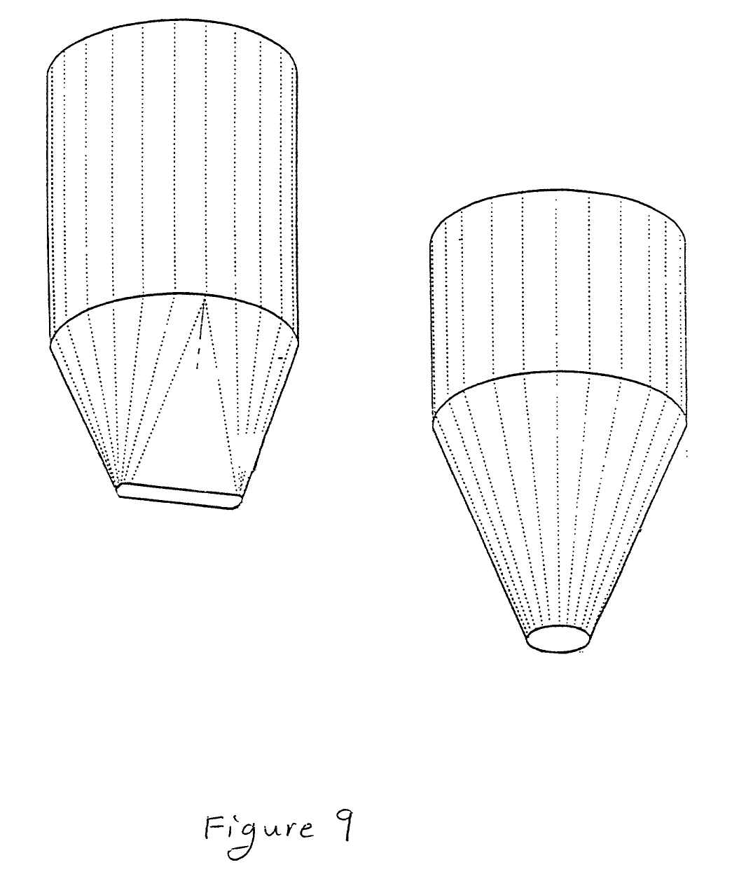

In addition, maximum hopper angles for mass flow were made utilizing the test method of ASTM D6128-97. Results are indicated in Tables 3 and 4: The relationship of the various angles to typical bin dimensions are indicated in FIG. 9.

In one embodiment, the present invention comprises an apparatus for transporting thyroid hormone drug formulations from a blender to a tableting machine and further comprising a blender discharge section, a portable container section, a portable container discharge section, a conical tableting machine inlet section that utilizes bulk mass flow. In order to achieve bulk mass flow, each section of the invention is designed and fabricated for mass flow.

In general, all interior sloping surfaces of the present invention are preferably polished to an average roughness (R_{a) of} 10 microinch or better and more preferably to an average roughness of 3 microinch or better. Stainless steel sheet, grade 304 and 316, is available for fabrication purposes with a 2B finish which has a typical Rₐ of 5 to 20 microinch, depending upon the thickness of the sheet. It is further available in bright annealed or mirror finish with an average Rₐ of less than 1.0.

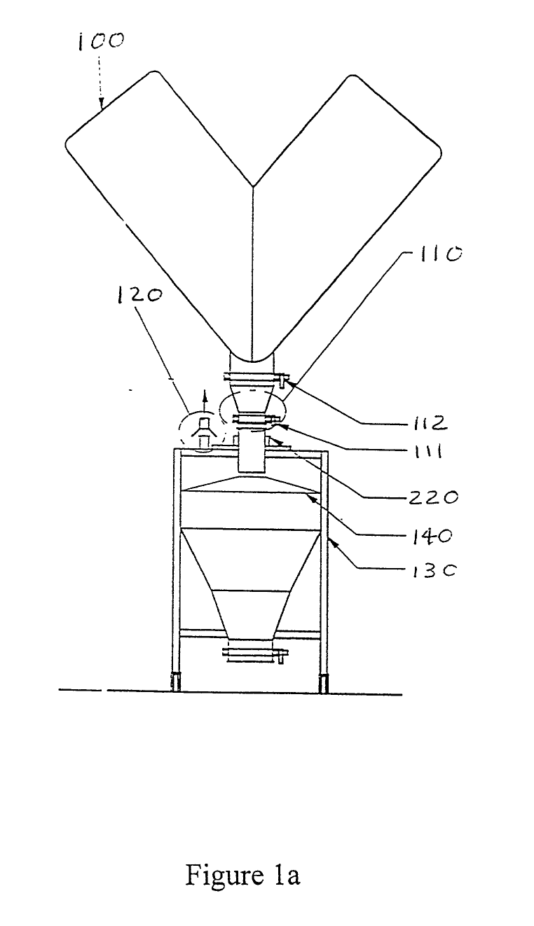

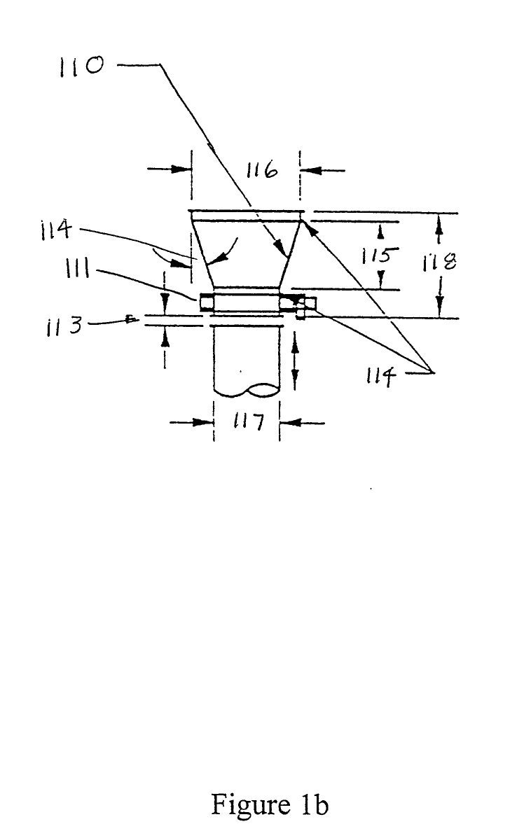

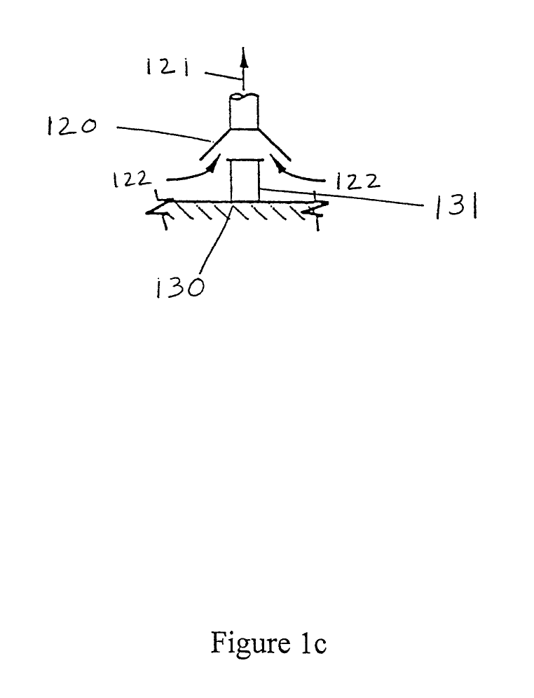

FIG. 1a illustrates one arrangement of the blender discharge section of a preferred embodiment of the present invention. Drug formulations are first blended in the inverted “V” blender 100. The formulation blend exits through a shut off valve 112, restriction section 110, restriction section shut off valve 111, and portable container 130 inlet section 220. The portable container 130 is further provided with an open vent 120. Shut off valves 111 and 112 are hygienic service valves, for example, as manufactured by COIRA, although other valve manufactures are acceptable. Valve 111 has an internal diameter matching that of blender 100 discharge. Valve 112 has an internal diameter matching that of the discharge of the restriction section 110. The shutoff valve 112 is optional and is not intended as a limitation to the present invention.

FIG. 1b illustrates one embodiment of the present invention restriction section 110. The restriction section is a conical restriction and reduces the blender 100 outlet 116 from 10 inches down to the dimensions of the 6 in. diameter shutoff valve 111. Valve 111 provides a more precise shutoff and better containment of residual material after transfer. This restriction section is intended to slow the discharge of the formulation blend so that it is less aerated upon filling into the container. This section will also allow for better filling accuracy, since the discharge rate (with proper venting) through a larger, for example 10 inch valve outlet, is expected to be very high. Flexible connections 114 connect the restriction section to the blender outlet shutoff valve. Suitable hygienic flexible seals are available from, for example Muller. Inlet diameter 116 matches blender 100 outlet dimension while outlet diameter 111 is determined to provide reliable flow with no ratholing or arching. The conical sidewall angle 114, restriction conical height 115 and overall height 118 are set to maintain bulk mass flow through the restriction section. One embodiment of the present invention uses a sidewall angle 113 of 17 degrees, height 115 of 6.5 inches and overall height 118 of 10 inches or less. Air gap 113 is approximately 1 inch. During discharge of blender 100, unrestricted venting at the top of blender must be maintained.

FIG. 7c illustrates an embodiment of an open vent 120 for portable container 130. Discharge 121 of vent hood 120 is connected to an appropriate dust collection system. Dust displaced from portable container 130 through vent port 131 along with ambient air 122 is withdrawn by the dust collection system 121. This vent Is intended to provide a path for air to escape during filling. A “hard” connection is not recommended for this use, as it may be too aggressive in removing dust. An open connection (as illustrated) will ensure that any displaced dust and air is removed by the dust collection system, A filter cartridge can be used in place of the vent. assuming it provides the necessary air flow rate and filtration level.

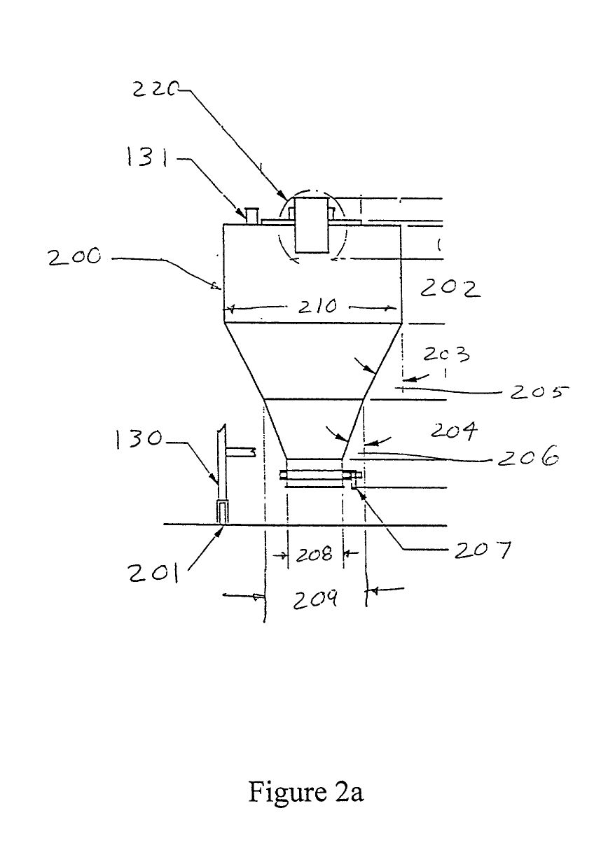

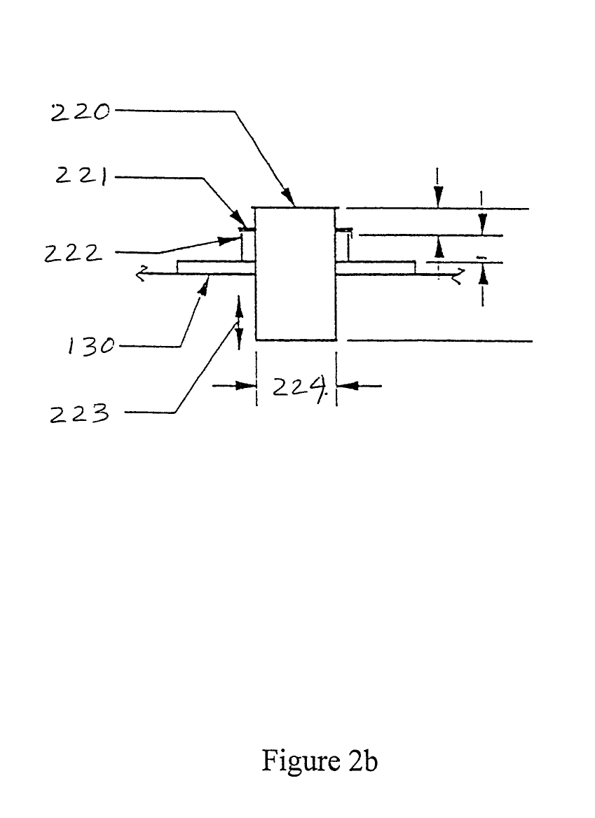

FIG. 2a illustrates an embodiment of a portable container 130 of the present invention. The portable container is supported by a frame further supported on casters 201 and comprises a storage container 200 with an upper cylindrical portion 202, middle conical section 203 and lower conical section 204. As has been previously illustrated in Tables 3 and 4, the maximum wall angle of a conical mass flow container is dependent upon the diameter of the container, with larger diameter containers having higher maximum angles (assuming wall roughness is constant). This being the case, a three section container can hold greater volumes for a given height by using a less steep wall angle in the larger diameter section. As an example, and not a limitation the following dimensions are given: upper section 200 has an internal dimension of approximately 32 inches, middle section 203 has a wall angle 205 of 26 degrees and lower section 204 has a wall angle 206 of 20 degrees with a vertical height of 11 inches, an upper internal diameter of 18 inches and a lower internal diameter to match the internal diameter of portable container shutoff valve 207, for example approximately 10 inches. Shut off valve 207 is a hygienic valve as described earlier an attached to the portable container with a flexible connector such as a Muller brand seal.

FIG. 2b illustrates one embodiment of a floating cylindrical connection 220to a portable container of the present invention. The upper portion of the vent rests on guard 227 that is supported by a seal 222 and can be adjusted in the vertical direction 223 so as to mate with the discharge of blender 100. The internal diameter 224 matches the restriction section outlet diameter, for example 6 inches.

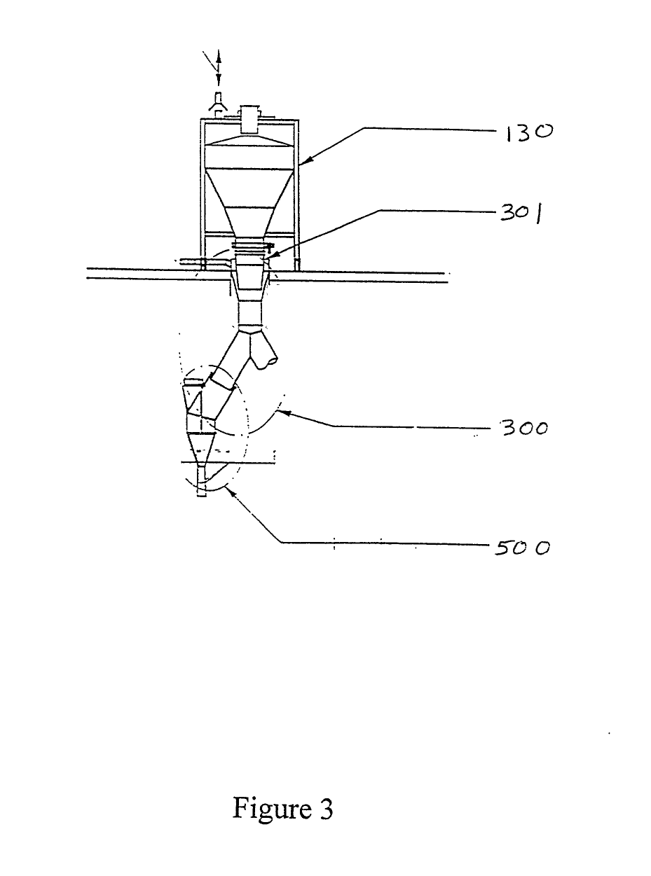

FIG. 3 shows the discharge of the portable container into the Y section 300 below. Note that air must be allowed to flow back into the container through the same top vent to ensure there is no counter flow through the material. A nested vent cone 301, shown beneath the container outlet, provides a path for displaced air to escape as well. In this case, a filter cartridge should be used, as even with an open connection the vacuum from a dust collection system may be too aggressive when the Y is full and result in product loss. Again, the cartridge must be sized to allow for the necessary air flow rate and filtration level. To provide easy access to the vent cone and valve discharge, the container outlet has been elevated within its framework compared to the surrounding floor (the separate action of elevating the container is not necessary, due to this design feature).

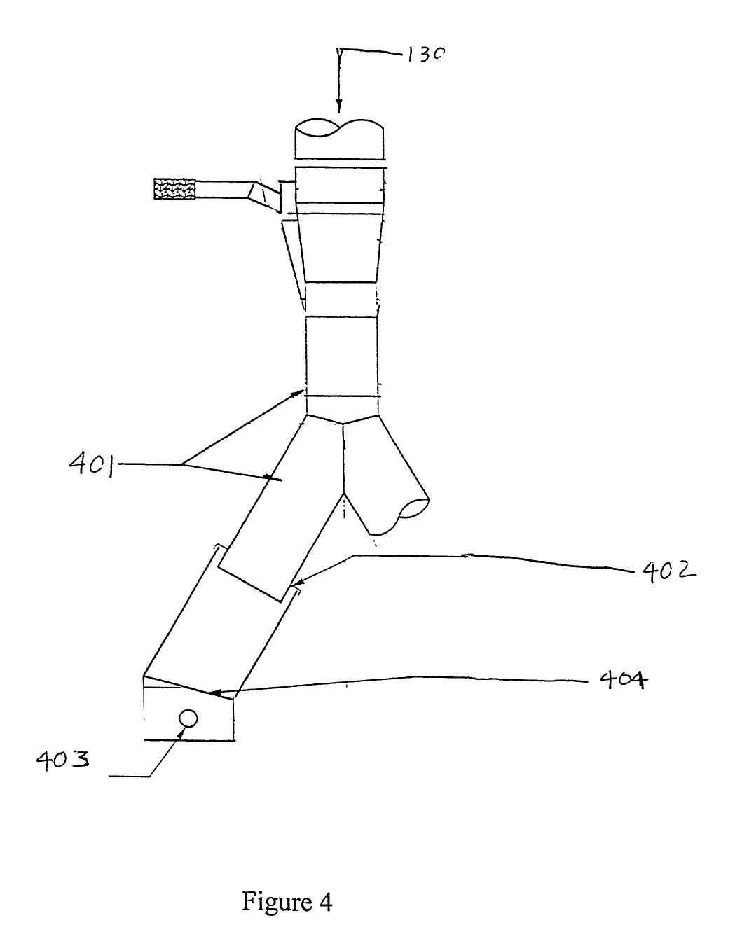

Details regarding one preferred embodiment of vent cone and Y are shown in FIG. 4. The constant diameter pipe sections 401 of a minimum 8 inch diameter should reliably convey the material without arching occurring. The welded joints between pipe sections 401 are preferably mitered joints 404, as illustrated. An expansion joint 402 will likely be required in each of the sloping legs of the Y. This joint will minimize any vibrations from the tableting machine, as well as any manufacturing or assembly tolerances, to not overly stress the system. This is an important feature to prevent possible pinching, and flow problems in the flexible portion of a connector between the tableting machine inlet and tableting machine inlet hopper. The joint could conceivably be formed through the use of a Muller-type insertion seal coupled with a smaller diameter upper section and a larger diameter lower section (as shown). As with the portable container, this design assumes all interior surfaces are electropolished 304 stainless steel sheet with an average roughness of 3.0 microinches or better.

The Y section 407 is needed when one portable container is to feed two tableting machines simultaneously and can be omitted when only a single tableting machine is present. An optional sight glass 403 may be provided as an aid to operating personnel. When so provided, it must be installed so as not to protrude into the flow channel. The outlet of the vent cone and Y-section discharges to the tableting machine inlet hopper.

Dimensions depicted on FIGS. 4, 5 and 6 are by way of illustration only and are not intended to be otherwise limitations to the present invention.

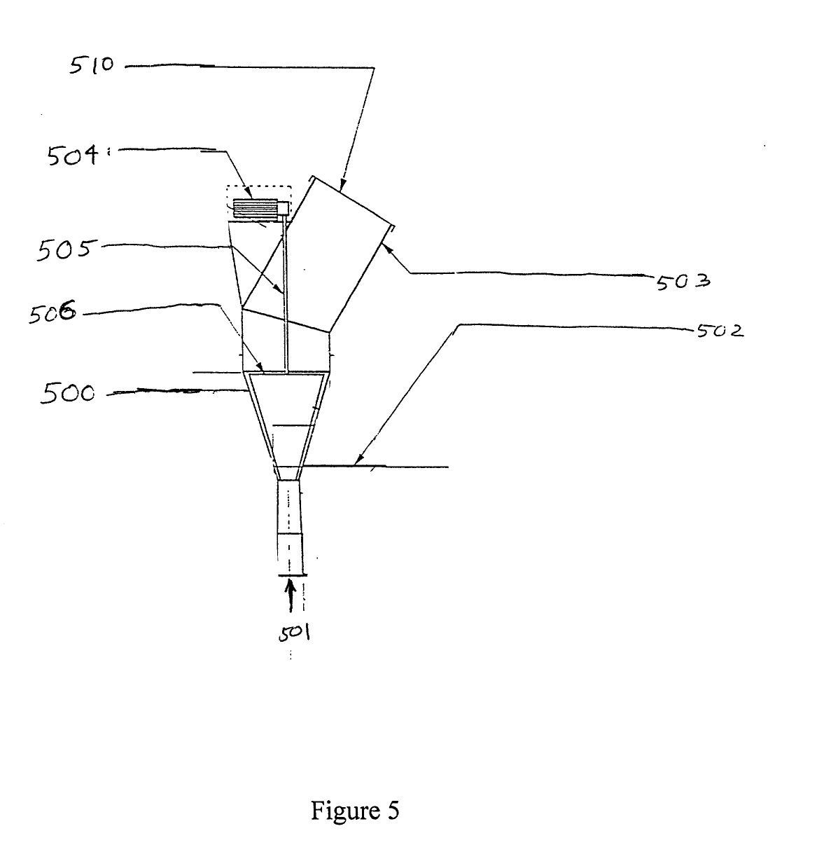

One embodiment of a tableting machine inlet feed hopper is shown on FIG. 5. This system consists of a conical mass flow hopper, that reduces to a slightly smaller (2½ in.) diameter at its base than the press feeder inlet. Mass flow is required to prevent ratholing and will be provided by the hopper slope (at 15 degrees from vertical) and interior surfaces (electropolished 304 stainless steel sheet) shown. The extra reduction (below the 80 mm final outlet size) will allow for a small degree of divergence to be used within a lower extension that terminates at the press feeder inlet. Divergence is recommended for this vertical section as it will provide a significant reduction in arching potential. The overall hopper shape and height can be chosen to minimize the physical impact when fitting to an existing tableting machine. For example, the existing cutout and clamping system for a prior tableting machine can be used without any changes to the machine. The conical mass flow hopper has a flange plate, to help locate the hopper on the machine.

The tableting machine feed hopper contains agitation, as shown in FIG. 5, to prevent the formulation blends from arching and thereby forming a blockage within it. Various methods of reducing the arching potential are useful. In a preferred embodiment, an agitator system is selected as providing better long term reliability and capability of feeding consistent and uniform material. The agitator blades should extend from the hopper inlet all the way to the 2½ in. diameter outlet. The agitator drive shaft extends up and inside the the Y-bottom section and out the top of the inclined pipe through a seal. In addition, the agitator drive shaft may be coupled at various points to allow easy disassembly and cleaning. The inclined lower pipe of the Y may be provided in sections for these same reasons. The drive motor is placed outside the Y and is enclosed for protection and containment.

To provide the connections between the various pieces shown in FIGS. 1a through 6, flexible connections should be used. Insertion seals, such as those made by Muller are common in the industry, and provide acceptable sealing capability during normal operation. Another approach is used in which a feature of the stationary item (blender or discharge cone) provides some vertical displacement to mate with a container by rigid clamping.

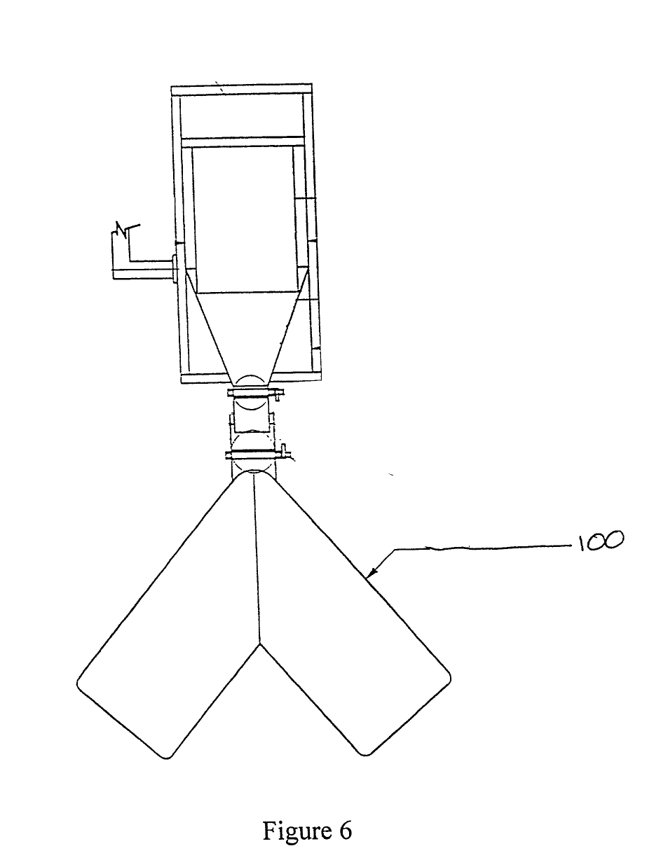

An arrangement for filling the blender from a drum inversion station is shown in FIG. 6. An interface piece, with an insertion seal, is attached beneath the blender discharge valve. The blender is inverted. with this interface attached, for filling. The drum cradle consists of a movable bottom that will force the top of the drum into the drum cone prior to being raised and inverted. Various drum sizes can be handled by such a system, depending on the cradle and drum cone size that is chosen, although care must be taken so that the drum cone is not scratched in sections that will have material contact. The drum cone is shown as being electropolished 304 stainless steel sheet with a 20 degrees (from vertical) cone angle in order to discharge material reliably. For other formulations, a steeper angle may be required. A hard stop is shown as being attached to the drum cone outlet to prevent it from being inserted too far into the blender interface piece and possibly interfering with its 10 in. valve.

Additional design considerations are incorporated into preferred embodiments of the present invention and include:

In a mass flow container, the hopper section must be sufficiently steep and low enough in friction to cause all the material to flow, without stagnant regions, whenever any material is withdrawn. “All the material is flowing” does not imply or require that all the particles are flowing at the same velocity. For example, particles which are in the converging hopper section flow at different velocities.

Material flows more slowly at the walls than at the centerline of the hopper, due to friction with the walls. This effect becomes visibly apparent when the material level is just above or within the hopper. It also becomes more pronounced here because of the lack of head pressure. In some cases, such a velocity differential may be beneficial (e.g. for inblending). In other applications. a uniform velocity may be required to minimize particle segregation effects, enforce uniform residence time, or provide a well defined transition from one material to another.

It is essential that such requirements be properly taken into account when designing a container. In addition to a proper design for a mass flow container, the quality of construction is critical. Protrusions into the flow channel caused by horizontal welds, incorrectly lapped liner plates or poorly constructed mating flanges will prevent mass flow.

Similarly, poor quality surface finish caused by weld spatter, poor quality workmanship or simply not using the liner material specified may prevent mass flow. Conditions below the outlet are just as important as the hopper design. Gates must allow the bulk solid to flow uniformly, and feeders must withdraw the bulk solid from the entire outlet area. It is imperative that the fabricator be made aware of both the design intent and the need for good quality workmanship. In addition, the engineer should carefully inspect the fabrication of the hopper and feeder.

For good interior surface finish, whenever possible, welding should be done on the outside of the hopper. If interior welding is necessary, all welds on sloping surfaces must be ground flush and power brushed to retain a smooth surface. After welding, all sloping surfaces must be clean and free of weld spatter. The surface finish is most critical in the region of the hopper outlet; therefore, any blisters from exterior welding in this area must be brushed smooth.

Horizontal or diagonal welded connections should preferably be lapped with the upper section on the inside so that the resulting ledge does not impede flow. If horizontal butt welds are used, care must be taken to avoid any protrusion into the flowing, solid. Vertical welds coinciding with the direction of material flow should preferably be butted, then ground flush and power brushed as noted above. Mating flanges: the lower of two mating flanges must be oversized to prevent any protrusions into the flowing solid. The amount of oversize depends on the accuracy of the construction and erection, usually one inch overall is sufficient.

All flanges should be attached to the outside of the hopper with the hopper wall material being the surface in contact with the flowing solids. This ensures that the flange does not protrude into the flowing solids. Feeder or gate below hopper: Either a feeder, a cutoff gate or both may be used below the hopper outlet. The key to feeder and gate design is to provide uniform withdrawal of the bulk solid from the entire area of the outlet. If a gate is used below a mass flow hopper, the gate must be either fully open or fully closed. A partially opened gate creates a flow obstruction and will convert what would otherwise be a mass flow design into funnel flow.

It is equally important that the gate be selected carefully to ensure that the actual opening size is larger than the container outlet opening. Unless a full-port design is specified, the port size of the valve may be significantly smaller than the nominal valve size. As an example, the actual port openings for typical metal seated and elastomer seated 12 in. knife gates are, respectively, 11 in. and 10 in. Therefore, even if the outlet of a mass flow container designed with a nominal outlet diameter of 12 inches were undersized by a full inch to 11 inches, it would still be too large for the elastomer seated valve. This example emphasizes the importance of checking the valve specifications and sizing the valve and outlet accordingly. Modulation of flow rate is preferably accomplished with a feeder, not a gate.

In general, clean out ports such as poke holes and rod-out ports are not recommended in mass flow container designs, as they have a tendency to prevent flow along the walls, thus creating a problem that mass flow containers are intended to solve. Access doors are also a frequent cause of problems. If they are essential, it is better to locate them in the cylinder rather than in the hopper section.

Stainless steel plate and sheet can be obtained in a variety of surface finishes. Generally. a given finish is smoother for sheet thicknesses (10 gauge or thinner) than for plate thicknesses 3/16 inch or thicker). Some finishes , such as 2B, may be available only in sheet thicknesses.

While this invention has been described in terms of manufacturing thyroid hormone preparations, it should be evident that the present invention is applicable to the manufacture of pharmaceutical tablets in general and other tablets or dry formulations where the consistency of active ingredient concentration is important.

1. an apparatus for transporting thyroid hormone drug formulations from a blender to a tableting machine comprising:
a blender discharge section;
a portable container section;
a portable container discharge section;
and a conical tablet press inlet section;
wherein mass flow of the drug formulation is maintained in all sections of the apparatus.

2. The apparatus of claim [c1], [Claim Reference]
wherein the portable container discharge section comprises a vent cone section.

3. The apparatus of claim [c1], [Claim Reference] wherein the portable container discharge section comprises a vent cone section and a Y-branch section.

4. The apparatus of claim [c1], [Claim Reference] wherein the thyroid drug formulation comprises one or more active moieties selected from the group consisting of levothyroxine sodium and liothyronine sodium.

5. The apparatus of claim [c1], [Claim Reference] wherein the thyroid drug formulation comprises the active moiety liothyronine sodium, calcium sulfate, gelatin, starch, stearic acid, sucrose and talc.

6. The apparatus of claim [c1], [Claim Reference] wherein the thyroid drug formulation comprises the active moiety levothyroxine sodium, lactose, microcrystalline cellulose, pregelatinized starch, and magnesium stearate.

7. The apparatus of claim [c1], [Claim Reference] wherein the thyroid drug formulation comprises the active moiety levothyroxine sodium and inert ingredients suitable for treating canine hypothyroidism.

8. The apparatus of claim [c1], [Claim Reference] wherein the thyroid drug formulation comprises the active moiety levothyroxine sodium and inert ingredients suitable for treating feline hypothyroidism.
